(19) United States
(12) Patent Application Publication
(10) Pub. No.: US 2015/0232458 A1
(43) Pub. Date: Aug. 20, 2015

(54) PROCESS FOR PREPARATION OF TRIGLYCIDYL ISOCYANURATE (TGIC)

(71) Applicant: PIRAMAL ENTERPRISES LIMITED, Mumbai (IN)

(72) Inventors: Pravin PATIL, Maharashtra (IN); Samir BENDRE, Thane (IN); Ashutosh JAGTAP, Mumbai (IN); Mita ROY, Mumbai (IN)

(73) Assignee: PIRAMAL ENTERPRISES LIMITED, Mumbai (IN)

(21) Appl. No.: 14/627,055

(22) Filed: Feb. 20, 2015

(30) Foreign Application Priority Data
Feb. 20, 2014 (IN) 594/MUM/2014

(51) Int. Cl. C07D 405/14

(52) U.S. Cl. CPC C07D 405/14

(57) ABSTRACT

The present invention relates to an improved process for the preparation of 1,3,5-triazine-2,4,6(1H,3H,5H)-trione (“Triglycidylisocyanurate (TGIC) or the compound of formula I”) comprising reacting cyanuric acid (the compound of formula III) with 3 to 7 molar equivalents of epichlorohydrin in an autoclave at a temperature of 80-100° C. for 1 hour to give the mixture of intermediates A, B and C and reacting intermediates A, B and C with an alkali to obtain the compound of formula I.

### FIELD OF THE INVENTION

The present invention provides an improved process for the preparation of a key intermediate, 1,3,5-triazine-2,4,6(1H,3H,5H)-trione (referred to herein as “Triglycidyl isocyanurate (TGIC) or the compound of formula I”) which is useful for the synthesis of 5-[(3,5-dimethylphenoxy)methyl]-1,3-oxazolidin-2-one (Metaxalone or the compound of formula II); a muscle relaxant.

### BACKGROUND OF THE INVENTION

Metaxalone, 5-[(3,5-dimethylphenoxy)methyl]-1,3-oxazolidin-2-one is represented by following formula II (hereinafter referred as “the compound of formula II”).

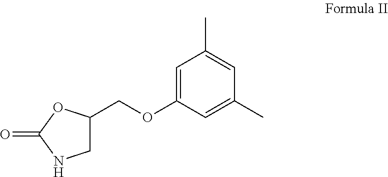 [SMILES: Cc1cc(C)cc(OCC2CNC(=O)O2)c1]

Metaxalone is a muscle relaxant used to relax muscles and relieve pain caused by strains, sprains, and other musculoskeletal conditions. Metaxalone is marketed by King Pharmaceuticals under the brand name SKELAXIN®. SKELAXIN® is available in an 800 mg scored tablet.

Triglycidyl isocyanurate (TGIC), chemically known as 1,3,5-triazine-2,4,6(1H,3H, 5H)-trione, is an epoxy compound, which is represented by the following formula I (alternatively referred to as “the compound of formula I”).

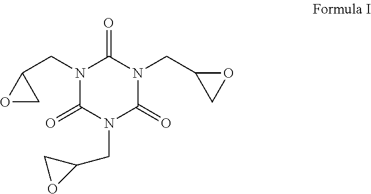 [SMILES: O=c1n(CC2CO2)c(=O)n(CC2CO2)c(=O)n1CC1CO1]

Triglycidyl isocyanurate (TGIC or the compound of formula I) contains three epoxide groups which give alkylating and cross-linking properties to the chemical. Triglycidyl isocyanurate (TGIC), in its molten state reacts easily with various functional groups in the presence of catalysts or promoters. Triglycidyl isocyanurate (TGIC), like other epoxides, can react with amines, carboxylic acids, carboxylic acid anhydrides, phenols and alcohols.

Triglycidyl isocyanurate (TGIC) is a key intermediate useful in the synthesis of metaxalone; a muscle relaxant. It is also used as a cross-linker in the manufacture of laminated sheets, printed circuits, electrical insulations, inks, adhesives and lining materials.

A process for the preparation of the compound of formula I is described in U.S. Pat. No. 2,809,942 (U.S.'942 Patent). The process of U.S.'942 Patent involves the reaction of cyanotic acid (hereinafter referred as “the compound of formula III”) with epichlorohydrin in the presence of 60% aqueous solution of benzyl trimethyl-ammonium chloride at 105-110° C. for 3 hours. After completion of the reaction, the reaction mixture is cooled to 38° C. To the reaction mixture, sodium hydroxide is added and excess of epichlorohydrin is removed by distillation at 98° C. To the reaction mixture, acetone is added at room temperature. The reaction mixture is filtered to remove the inorganic solid and the filtrate is collected. To the filtrate, sodium hydroxide is added again and the resulting reaction mixture is then filtered to remove inorganic solid. The filtrate is collected and distilled under vacuum to obtain the compound of formula I. It is observed that the process disclosed in said patent requires use of 15 molar equivalents of epichlorohydrin per mol of the compound of formula III. Epichlorohydrin is included in group 2A list of carcinogens by IARC (International Agency for Research on Cancer), intergovernmental agency forming part of the World Health Organisation of the United Nation. Therefore, handling of such hazardous reagent in an open system in excess amount is difficult on a commercial scale. Moreover, epichlorohydrin being a costly reagent, use of the same in excess amount renders the whole process costly. Further, the product, the compound of formula I obtained by the said process contains epichlorohydrin as an impurity.

Another patent document, Indian patent application no. 1082/MUM/2004 describes a process for the preparation of the compound of formula I. The process involves addition of the compound of formula III to the aqueous solution of benzyl trimethyl-ammonium chloride to obtain a reaction mixture. To the reaction mixture, epichlorohydrin is added and the reaction mixture is heated at 95-100° C. for 60 minutes. After completion of the reaction, the excess epichlorohydrin is distilled under vacuum. To the reaction mixture, sodium hydroxide and dichloromethane are added under stirring. The reaction mixture is filtered and the solution is collected. To the collected solution, methanol is added at 15° C. to precipitate the solid. The precipitated solid is filtered and dried to obtain the compound of formula I. The process disclosed in said patent application involves addition of sodium hydroxide in single lot to the reaction mixture. Addition of sodium hydroxide in single lot to the reaction mixture generates high exotherm. Due to this high exotherm, there is rapid rise in the temperature and pressure with a possibility of explosion and fire. Similar to the process described in U.S. Pat. No. 2,809,942, the process described in this patent application also involves use of 15 moles of epichlorohydrin per mol of the compound of formula III. Thus, process described in this patent application is not industrially viable.

U.S. Pat. No. 3,910,908 (U.S.'908 Patent) described a process for the preparation of the compound of formula I by reaction of the compound of formula III with epichlorohydrin. The U.S.'908 Patent indicated that in order to achieve the compound of formula I in good yield and purity, it is necessary to use minimum 9 moles of epichlorohydrin per mole of the compound of formula III and the preferred proportion is 9 to 15 moles per mole of the compound of formula III. The reaction of the compound of formula III with epichlorohydrin is highly exothermic. The adiabatic temperature rise in this reaction is 135° C. Also, addition of sodium hydroxide to the reaction mixture generates high exotherm. Effective management of the released heat is critical for safe scale-up. Further the processes disclosed in the prior art involves the use of epichlorohydrin in excess amount. Handling of epichlorohydrin in excess amount is difficult in an open system. Therefore, it is required to minimise the quantity of epichlorohydrin used and avoid the contact with epichlorohydrin vapours, which would require engineering design parameters such as temperature jacket, heat exchangers or condensers, external cooling system etc.

### OBJECT OF THE INVENTION

An object of the present invention is to provide an improved process for the preparation of the compound of the formula I (triglycidyl isocyanurate (TGIC)) comprising reacting the compound of formula III with epichlorohydrin in a closed system such as an autoclave.

Another object of the present invention is to provide an improved process for the preparation of the compound of the formula I comprising using 3 to 7 molar equivalents of epichlorohydrin relative to the compound of formula III.

Another object of the present invention is to provide an improved process for the preparation of the compound of the formula I in a cost effective manner.

Yet another object of the present invention is to provide an improved process for the preparation of the compound of formula I by avoiding the contact with toxic vapors of epichlorohydrin thereby rendering the process safe and environment friendly.

Further object of the present invention is to provide an improved process for the preparation of the compound of formula I by reducing the chances of explosion and fire thereby rendering the process safe and industrially viable.

### SUMMARY

In accordance with an aspect of the present invention, there is to provided an improved process for the preparation of the compound of formula I (triglycidylisocyanurate (TGIC)) comprising the steps of,

a. reacting the compound of formula III with 3 to 7 molar equivalents of epichlorohydrin using a phase transfer catalyst (PTC) in an autoclave at a temperature of 80-100° C. for 1 hour to obtain a reaction mixture;

b. extracting the reaction mixture of the step (a) with a chlorinated solvent and adding alkali lot wise to the chlorinated solvent at 10-15° C. to obtain the compound of formula I.

In the context of the present invention, the reaction mixture obtained in the step (a) contains a mixture of intermediates A, B and C; which are structurally represented below.

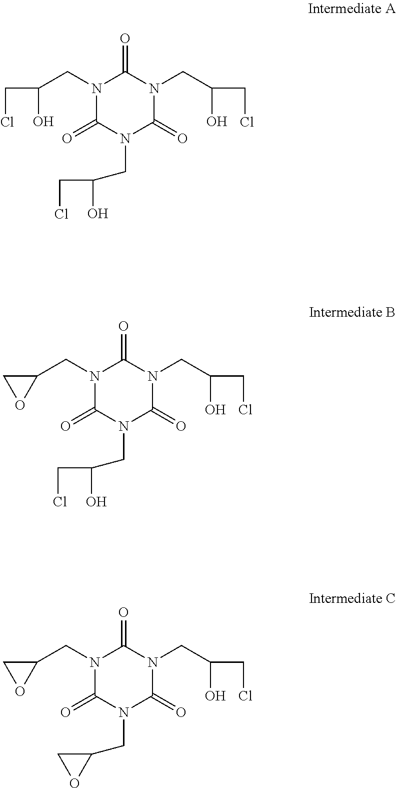 [SMILES: O=c1n(CC(O)CCl)c(=O)n(CC(O)CCl)c(=O)n1CC(O)CCl ; O=c1n(CC(O)CCl)c(=O)n(CC2CO2)c(=O)n1CC(O)CCl ; O=c1n(CC(O)CCl)c(=O)n(CC2CO2)c(=O)n1CC1CO1]

In summary, according to present invention, the process for the preparation of the compound of formula I involves the reaction of the compound of formula III with 3 to 7 molar equivalents of epichlorohydrin in the presence of a phase transfer catalyst (PTC) in an autoclave to obtain the mixture of intermediates A, B and C. In the second step, dehydrohalogenation of intermediates A, B and C occurs in the presence of an alkali to obtain the compound of formula I.

The process of the present invention is depicted in the following scheme-I.

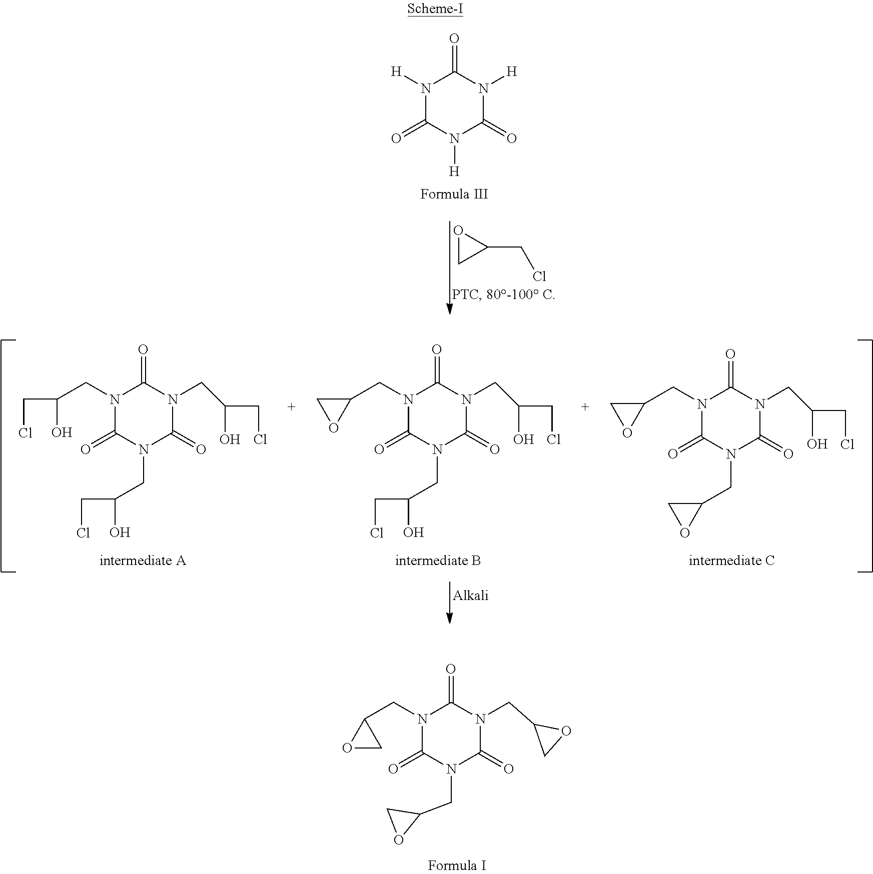

### DETAILED DESCRIPTION OF TOE INVENTION

The present invention relates to an improved process for the preparation of the compound of formula I (triglycidylisocyanurate (TGIC));

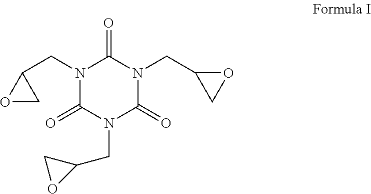 [SMILES: O=c1n(CC2CO2)c(=O)n(CC2CO2)c(=O)n1CC1CO1]

comprising,

a. reacting the compound of formula III (cyanotic acid),

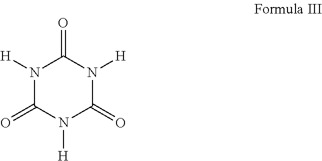

with 3 to 7 molar equivalents of epichlorohydrin using a phase transfer catalyst (PTC) in an autoclave at a temperature ranging from 80 to 100° C. for 1 hour to obtain a reaction mixture;

b. extracting the reaction mixture of the step (a) with a chlorinated solvent and adding alkali lot wise at a temperature ranging from 10-15° C. to the chlorinated solvent to obtain the compound of formula I.

In accordance with an embodiment of the present invention, epichlorohydrin used in the step (a) of the process is in an amount ranging from 4 to 6 molar equivalents based on the compound of formula III.

In accordance with an embodiment of the present invention, the phase transfer catalyst used in the step (a) of the process is selected from the group consisting of tri-n-propyl amine, triethanolamine, methyl triethyl ammonium chloride and benzyl trimethyl ammonium chloride.

In specific embodiment of the present invention, the phase transfer catalyst used in the step (a) of the process is benzyl trimethyl ammonium chloride.

In accordance with an embodiment of the present invention, the phase transfer catalyst used in the step (a) of the process is in an amount ranging from 0.1 to 0.15 molar equivalents based on the compound of formula III.

In accordance with an embodiment of the present invention, the reaction mixture obtained in the step (a) of the process contains a mixture of intermediates A, B and C as described above.

In another embodiment of the present invention, the intermediates A, B and C obtained in the step (a) are further converted insitu into the compound of formula I in the step (b) by addition of an alkali.

In accordance with another embodiment of the present invention, the chlorinated solvent used in the step (b) of the process is selected from the group consisting of dichloromethane (DCM), chloroform and carbon tetrachloride.

In specific embodiment of the present invention, the chlorinated solvent used in the step (b) of the process is dichloromethane (DCM).

In accordance with another embodiment of the present invention, in the step (b) of the process, the chlorinated solvent used is in an amount ranging from 10 to 15 volumes based on the compound of formula III.

In accordance with another embodiment of the present invention, wherein in the step (b) of the process, alkali is added to the chlorinated solvent lot wise over a period of 4 hours.

In accordance with another embodiment of the present invention, alkali used in the step (b) of the process is selected from the group consisting of sodium hydroxide, potassium hydroxide and calcium hydroxide.

In accordance with another embodiment of the present invention, in the step (b) of the process, alkali is used in an amount ranging from 3 to 4 molar equivalents based on the compound of formula III.

The compound of formula I obtained by the process of the present invention is further purified by using an alcohol to obtain the pure compound of formula I (triglycidylisocyanurate (TGIC)).

The alcohol used for purification of the compound of formula I is selected from the group consisting of methanol, ethanol and isopropyl alcohol.

The starting material of the process i.e. the compound of formula III is a known compound and can be prepared by a person skilled in the art by using methods known in the art such as those described in the literature. For example, a process described in the U.S. Pat. No. 2,872,447 can be used for the preparation of the compound of the formula III. The process involves heating of urea in dimethylformamide (DMF) for 6 hours at a reflux temperature. After 1.5 hours of heating, the compound of formula III begins to precipitate. The resulting precipitate is then cooled and filtered. The precipitate is then washed with cold water and dried at 105° C. to obtain the compound of formula III.

According to an embodiment of the present invention; the process for the preparation of the compound of formula I involve the reaction of the compound of formula III with epichlorohydrin in the presence of aqueous benzyl trimethyl ammonium chloride as the phase transfer catalyst in an autoclave for 1 hour at 85-100° C. to provide a reaction mixture. The resulting reaction mixture contains a mixture of intermediates A, B and C which was cooled to room temperature and extracted with DCM. The DCM layer was further cooled to 10-15° C. To the DCM, sodium hydroxide was added lot wise over a period of 4 hours. The resulting reaction mixture was further stirred for 1 hour at 10-15° C. to precipitate solid. The precipitated solid was filtered as an inorganic waste and solution was collected. The collected solution was distilled under vacuum to obtain the crude compound of formula I. The crude compound of formula I was further purified with methanol to obtain the pure compound of formula I.

The process for preparation of the compound of formula I involves the use of epichlorohydrin as one of the reagent. As discussed above, epichlorohydrin is carcinogenic in nature. The use of such a hazardous reagent in large scale, at high temperature and in open system may cause health hazards. Therefore, it is advisable to avoid extensive contact with vapours or liquid form of epichlorohydrin. In view of this, it is advisable to use epichlorohydrin in lower quantity. Also, the reaction of epichlorohydrin with the compound of formula III is highly exothermic. Due to this high exotherm, there is sudden increase in temperature and pressure and therefore, carrying out such a reaction in an open system may cause explosion and fire. Owing to such risk, it is highly prudent to carry out the reaction using a properly designed, leak-tight product handling systems and the like.

Our scientists have observed that the use of closed system such as an autoclave avoids the risk specified above as well as lead to simplicity in the operations. Autoclave has provided pressure regulator to maintain pressure and overpressure protection is done by safety valves. Therefore, high heat and pressure generated during the reaction of the compound of formula III with epichlorohydrin is easily managed by the use of autoclave. Further, vapour deposition of epichlorohydrin in the closed reactor (autoclave) itself avoids the losses of epichlorohydrin which consequently minimized the excess consumption and avoids unnecessary contact with toxic vapours unlike when the reaction is carried out in the open system.

Therefore, the process of the present invention directed to the preparation of the compound of formula I i.e. triglycidylisocyanurate (TGIC) has clear advantages in terms of reducing the excess consumption of raw materials and energy; minimising the contact with hazardous substances and resulting lower emissions and waste.

The following examples which fully illustrate the practice of the preferred embodiments of the present invention are intended to be for illustrative purpose only and should not be construed in any way to limit the scope of the present invention.

### EXAMPLE-I

To an autoclave, the compound of formula III (100 g), epichlorohydrin (350 ml), benzyl trimethyl ammonium chloride (17.5 g) and water (100 ml) were charged and the reaction mixture was stirred at a temperature of 75-80° C. for 1 to 2 hours. The resulting reaction mixture was then cooled to the temperature of 25-30° C. To the reaction mixture, then dichloromethane (DCM) (1000 ml) was charged and the reaction mixture was stirred for 30 min at a temperature of 25-30° C. The two layers formed were separated. The separated aqueous layer was again extracted with DCM. The DCM layers were collected and combined. To the combined DCM layer, sodium hydroxide (100 g) was charged in four lots. The reaction mixture was then filtered. The filtrate was then distilled under vacuum at a temperature of 50-55° C. and then to 80-85° C. to obtain an oil. To the obtained oil, methanol (400 ml) was added at 45-50° C. and the reaction mixture was heated at 60-65° C. The reaction mixture was then cooled to 0-5° C. to precipitate the solid. The precipitated solid was then filtered and washed with methanol. The filtered solid was dried under vacuum at 55-60° C. to obtain the compound of formula I in yield of 67% and purity of 98%.

### EXAMPLE-II

To an autoclave, the compound of formula III (500 g), epichlorohydrin (1750 ml), benzyl trimethyl ammonium chloride (85 g) and water (500 ml) were charged and the reaction mixture was stirred at a temperature of 75-80° C. for 1 to 2 hours. The resulting reaction mixture was then cooled to a temperature of 25-30° C. To the reaction mixture, then DCM (5000 ml) was charged and the reaction mixture was stirred for 30 minutes at a temperature of 25-30° C. The two layers formed were separated. The separated aqueous layer was again extracted with DCM (1000 ml). The DCM layers were collected and combined. To the combined DCM layer, sodium hydroxide (542.5 g) was charged in four lots. The reaction mixture was then filtered. The filtrate was then distilled under vacuum at a temperature of 50-55° C. and then to 80-85° C. to obtain oil. To the obtained oil, then methanol (2000 ml) was added at 45-50° C. and the reaction mixture was heated at 60-65° C. The reaction mixture was then cooled to 0-5° C. to precipitate the solid. The precipitated solid was then filtered and washed with methanol (500 ml). The filtered solid was dried under vacuum at 55-60° C. to yield the compound of formula I in yield of 67% and purity of 98%.

Details for HPLC analysis:

Instrument: Shimadzu LC-8A HPLC with SPD-10A VP PDA detector or equivalent.

Column: Lichrospher 100 RP18e (250 mm×4.0 mm) (5 μm) (P/N 1.50995.0001)

Detector: 210 nm

Column temperature: 40° C.

Flow rate: 0.5 ml/min.

Injection volume: 10 μl

Mobile phase: A: Water
B: Acetonitrile

Diluent: Acetonitrile:Water (50:50 v/v)

Run time: 35 minutes

### Blank Solution: Use Diluent

### Assay:

For system suitability, inject (10 μl) of the standard solution (6 replicates) into the chromatograph. Relative standard deviation for TGIC (the compound of formula I) peak in standard solution should not be more than 2.0%.

Inject (10 μl) of blank solution and sample solution (2 replicates) into the chromatograph, calculate the assay by the formula given below.

$$\frac{Mean Sample Area}{Mean Standard Area} \times \frac{Standard wt}{Sample wt} \times TGIC STD Potency$$

### Purity:

Inject (10 μl) of blank, reference standard and sample solution, measure the responses of all the peaks. In the sample chromatogram disregard any peak due to the blank. Confirm and identify peaks of TGIC (the compound of formula I) and cyanuric acid (the compound of formula III).

Calculation: Area %

Informative retention time:

Tris(2,3-epoxypropyl)isocyanurate (TGIC) (the compound of formula I): about 10.0 min

Cyanuric acid (the compound of formula III): ˜about 4.1 min

1. A process for the preparation of a compound of formula I (trigiycidylisocyanurate (TGIC)); 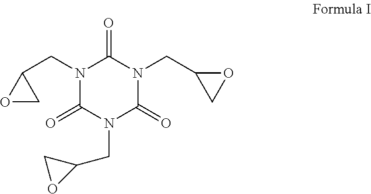 [SMILES: O=c1n(CC2CO2)c(=O)n(CC2CO2)c(=O)n1CC1CO1]
comprising,
a. reacting the compound of formula III, 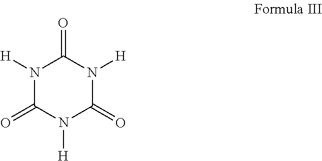
with 3 to 7 molar equivalents of epichlorohydrin using a phase transfer catalyst (PTC) in an autoclave at a temperature ranging from 80 to 100° C. for 1 hour to obtain a reaction mixture;
b. extracting the reaction mixture of the step (a) with a chlorinated solvent and adding an alkali lot wise to the chlorinated solvent at a temperature ranging from 10-15°C. to obtain the compound of formula I.

2. The process as claimed in claim 1, wherein in the step (a), epichlorohydrin is used in an amount ranging from 4 to 6 molar equivalents based on the compound of formula III.

3. The process as claimed in claim 1, wherein in the step (a), the phase transfer catalyst used is selected from the group consisting of tri-n-propyl amine, triethanolamine, methyl triethyl ammonium chloride and benzyl trimethyl ammonium chloride.

4. The process as claimed in claim 1, wherein in the step (a), the phase transfer catalyst used is benzyl trimethyl ammonium chloride.

5. The process as claimed in claim 1, wherein in the step (a), the phase transfer catalyst is used in an amount ranging from 0.1 to 0.15 molar equivalents based on the compound of formula III.

6. The process as claimed in claim 1, wherein in the step (a), the reaction mixture contains a mixture of intermediates A, B and C as structurally represented below.

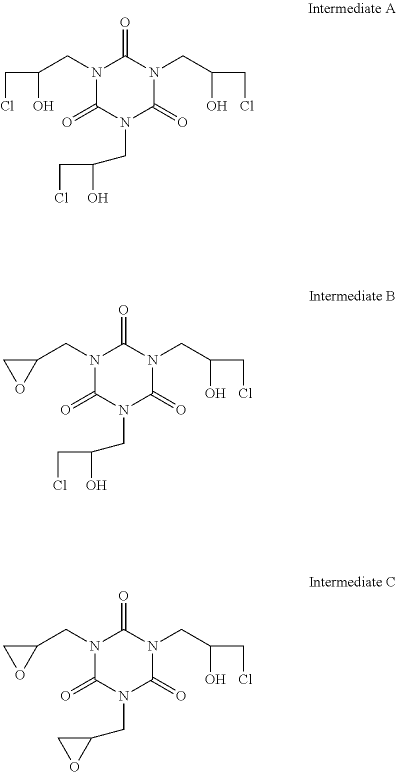 [SMILES: O=c1n(CC(O)CCl)c(=O)n(CC(O)CCl)c(=O)n1CC(O)CCl ; O=c1n(CC(O)CCl)c(=O)n(CC2CO2)c(=O)n1CC(O)CCl ; O=c1n(CC(O)CCl)c(=O)n(CC2CO2)c(=O)n1CC1CO1]

7. The process as claimed in claim 1, wherein in the step (b), the chlorinated solvent used is selected from the group consisting of dichloromethane (DCM), chloroform and carbon tetrachloride.

8. The process as claimed in claim 1, wherein in the step (b), the chlorinated solvent used is dichloromethane (DCM).

9. The process as claimed in claim 1, wherein in the step (b), the chlorinated solvent is used in an amount ranging from 10 to 15 volumes based on the compound of formula III.

10. The process as claimed in claim 1, wherein in the step (b), the alkali used is selected from the group consisting of sodium hydroxide, potassium hydroxide, calcium hydroxide and magnesium hydroxide.

11. The process as claimed in claim 1, wherein in the step (b), the alkali is used in an amount ranging from 3 to 4 molar equivalents based on the compound of formula III.

12. The process as claimed in claim 1, further comprises the purification of the compound of formula I using an alcohol selected from the group consisting of methanol, ethanol and isopropyl alcohol.

13. The process as claimed in claim 3, wherein in the step (a), the phase transfer catalyst used is benzyl trimethyl ammonium chloride.

14. The process as claimed in claim 7, wherein in the step (b), the chlorinated solvent used is dichloromethane (DCM).
